(19) United States
(12) Patent Application Publication
(10) Pub. No.: US 2025/0101076 A1
(43) Pub. Date: Mar. 27, 2025

(54) GROWTH HORMONE RECEPTOR ANTAGONISTS EXHIBITING INCREASED RECEPTOR BINDING PROPERTIES AND INCREASED SERUM HALF-LIFE

(71) Applicant: Molecular Technologies Laboratories LLC, Galena, OH (US)

(72) Inventors: Richard S. Brody, Galena, OH (US); Thomas J. Zupancic, Galena, OH (US); Uday Sandbhor, Galena, OH (US)

(21) Appl. No.: 18/402,090

(22) Filed: Jan. 2, 2024

Related U.S. Application Data
(60) Provisional application No. 63/539,874, filed on Sep. 22, 2023.

(51) Int. Cl. C07K 14/61; A61K 38/27

(52) U.S. Cl. CPC C07K 14/61; A61K 38/27

(57) ABSTRACT

Growth hormone receptor antagonists comprising human growth hormone receptor antagonist G120K, wherein a single amino acid of the human growth hormone receptor antagonist G120K has been mutated to cysteine, and wherein the single amino acid mutated to cysteine is T142; and a polydispersed 40 kDa branched polyethylene glycol molecule conjugated to the substituted cysteine in the human growth hormone receptor antagonist G120K mutant.

This patent application claims the benefit of U.S. Provisional Patent Application Ser. No. 63/539,874 filed on Sep. 22, 2023 and entitled “Methods for Treating Alzheimer's Disease and Related Dementias Using Pegylated Growth Hormone Antagonists”, the disclosure of which is hereby incorporated by reference herein in its entirety and made part of the present U.S. utility patent application for all purposes.

### REFERENCE TO A SEQUENCE LISTING

A sequence listing in computer readable form (CRF) is on file. The sequence listing is in an XML file entitled 44989-0017-U1.xml created on Dec. 13, 2023 and is 17 KB in size. The sequence listing is incorporated by reference as if fully recited herein.

### BACKGROUND

The described invention relates in general to compositions for use as receptor antagonists, and more specifically to novel human growth hormone receptor antagonists that have the potential to be highly effective therapeutics.

Human growth hormone, also known as somatotropin or somatropin, is a peptide hormone that stimulates growth, cell reproduction, and regeneration in humans and other animals. Growth hormone is a type of mitogen that is specific only to certain kinds of cells and is a 191-amino acid, single-chain polypeptide that is synthesized, stored, and secreted by somatotropic cells within the lateral wings of the anterior pituitary gland. Acromegaly is a syndrome that results when the anterior pituitary gland produces excess human growth hormone (hGH) after epiphyseal plate closure at puberty. If hGH is produced in excess prior to epiphyseal plate closure, the result is gigantism (or giantism). A number of disorders may increase the pituitary's hGH output, although most commonly it involves a tumor called pituitary adenoma, derived from a distinct type of cell (somatotrophs). Acromegaly most commonly affects adults in middle age and can result in severe disfigurement, complicating conditions, and premature death if untreated. Because of its pathogenesis and slow progression, the disease is hard to diagnose in the early stages and is frequently missed for years until changes in external features, especially of the face, become noticeable.

A receptor is a protein molecule usually found embedded within the plasma membrane surface of a cell that receives chemical signals from outside the cell. When such chemical signals bind to a receptor, they cause some form of cellular/tissue response such as, for example, a change in the electrical activity of the cell. In this sense, a receptor is a protein molecule that recognizes and responds to endogenous chemical signals. An agonist, such as human growth hormone, is a chemical composition that binds to a receptor and activates the receptor to produce a biological response. Whereas an agonist causes an action, an antagonist blocks the action of the agonist and an inverse agonist causes an action opposite to that of the agonist. A receptor antagonist is a type of receptor ligand or drug that blocks or dampens agonist-mediated responses rather than provoking a biological response itself upon binding to a receptor. These compositions are sometimes called blockers and examples include alpha blockers, beta blockers, and calcium channel blockers. In pharmacology, antagonists have affinity but no efficacy for their cognate receptors, and binding will disrupt the interaction and inhibit the function of an agonist or inverse agonist at receptors. Antagonists mediate their effects by binding to the active (orthosteric) site or to other (allosteric) sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist-receptor binding. The majority of drug antagonists achieve their potency by competing with endogenous ligands or substrates at structurally defined binding sites on receptors. By definition, antagonists display no efficacy to activate the receptors they bind and antagonists do not maintain the ability to activate a receptor. Once bound, however, antagonists inhibit the function of agonists, inverse agonists, and partial agonists.

Growth hormone receptor antagonists such as the product pegvisomant (sold under the trademark SOMAVERT®) are used in the treatment of acromegaly. Such compositions are used if the tumor of the pituitary gland causing the acromegaly cannot be controlled with surgery or radiation and the use of somatostatin analogues is unsuccessful. Pegvisomant is typically delivered as a powder that is mixed with water and injected under the skin.

PEGylation is the process of both covalent and non-covalent amalgamation of polyethylene glycol (PEG) polymer chains to molecules and macrostructures, such as drugs, peptides, antibody fragments, or therapeutic proteins. PEGylation is routinely achieved by incubation of a reactive derivative of PEG with the target molecule and produces alterations in physiochemical properties, including changes in molecular size and molecular charge. These physical and chemical changes increase systemic retention of the therapeutic agent and can influence the binding affinity of the therapeutic moiety to the cell receptors and can alter the absorption and distribution patterns. The covalent attachment of PEG to a drug or therapeutic protein can also “mask” the agent from the host's immune system (i.e., reducing immunogenicity and antigenicity), and increase the hydrodynamic size (i.e., size in solution) of the agent which prolongs its circulatory time by reducing renal clearance. PEGylation can also provide water solubility to hydrophobic drugs and proteins.

PEGylation, by increasing the molecular weight of a molecule, can impart several significant pharmacological advantages over the unmodified form of the molecule, such as: (i) improved drug solubility; (ii) reduced dosage frequency, without diminished efficacy and with potentially reduced toxicity; (iii) extended circulating life; (iv) increased drug stability; and (v) enhanced protection from proteolytic degradation. PEGylated drugs also include the following commercial advantages: (i) opportunities for new delivery formats and dosing regimens; and (ii) extended patent life of previously approved drugs. PEG is a particularly attractive polymer for conjugation and the specific characteristics of PEG moieties relevant to pharmaceutical applications include: (i) water solubility; (ii) high mobility in solution; (iii) lack of toxicity and low immunogenicity; and (iv) altered distribution in the body.

The addition of high molecular weight polyethylene glycols (PEGs) to proteins has been previously shown to increase the in-vivo half-lives of these proteins by a size dependent decrease in elimination by the kidneys. The addition of PEGs also lowers the immunogenicity of the proteins and decreases aggregation and protease cleavage (Pasut and Vronese, 2012; and Parveen and Sahoo, 2006). Multiple known PEGylated proteins have been approved by the USFDA for therapeutic use, including hormones, cytokines, antibody fragments, and enzymes (Pasut, and Veronese, 2012; Alconcel et al., 2011; and Kling, 2013). Thus, there is an ongoing need for the further development of PEGylated therapeutics, particularly for use in the treatment of diseases that are responsive to the use of human growth hormone (hGH) receptor antagonists or other receptor antagonists.

Evidence from human and animal studies supports a role for growth hormone (GH) in carcinogenesis. The level of growth hormone receptor (GHR) expression in 60 cancer cell lines from nine types of human cancer: breast, CNS, colon, leukemia, melanoma, non-small cell lung, ovarian, prostate, and renal has been determined [5]. High GHR expression levels were obtained for most of the cell lines for all of the cancer types except colon and leukemia. The GHR expression levels of the melanoma cell lines were exceptionally high, nearly fifty-fold higher than in the panel as a whole. Three melanoma cell types were treated with hGH and in two of the three hGH increased proliferation and induced activation of STAT5 and mTOR. The effects of GH and a siRNA mediated GHR knockdown cell line on tumor progression and epithelial mesenchymal transition in human melanoma cells has been investigated [6]. This research indicated that hGH promoted and GHR knockdown attenuated tumor proliferation, migration and invasion. The effect of GHR knockdown on the sensitivity of human melanoma cells to chemotherapy has been investigated [7]. This research indicated that hGH upregulates multiple ABC transporters, increasing the EC50 of the melanoma drug vemurafenib. GHR knockdown, in contrast, significantly increases drug retention by melanoma cells, decreases cell proliferation, and increases drug efficacy. Accordingly, the use of certain hGH antagonists as cancer therapeutics would be beneficial.

Evidence that supports a role for activation of the prolactin (PRL) receptor by either prolactin or growth hormone in carcinogenesis also exists. For certain cancers, especially breast, endometrial, liver, and prostate cancers, it has been reported in the literature that the human prolactin receptor is upregulated along with the human growth hormone receptor [11,12] Patients with both receptors being upregulated often have worse survival outcomes, which indicates that a molecule that antagonizes both receptors may act as a cancer therapeutic. Recent in vitro studies have demonstrated that GHRAs are antagonists against both the GH receptor and the PRL receptor and exhibit potent anticancer biological efficacies against human cancer cell lines [13].

### SUMMARY

The following provides a summary of certain example implementations of the disclosed technology. This summary is not an extensive overview and is not intended to identify key or critical aspects or elements of the disclosed technology or to delineate its scope. However, it is to be understood that the use of indefinite articles in the language used to describe and claim the disclosed technology is not intended in any way to limit the described technology. Rather the use of “a” or “an” should be interpreted to mean “at least one” or “one or more”.

A first disclosed composition provides a growth hormone receptor antagonist comprising human growth hormone receptor antagonist hGH-G120K having a DNA sequence of SEQ ID NO: 3 and an amino acid sequence of SEQ ID NO: 4, wherein a single amino acid of the human growth hormone receptor antagonist hGH-G120K has been mutated to cysteine, and wherein the single amino acid mutated to cysteine is T142; and a polydispersed 40 kDa branched polyethylene glycol molecule conjugated to the substituted cysteine in the human growth hormone receptor antagonist G120K mutant, and wherein the human growth hormone receptor antagonist is encoded by a DNA molecule having the sequence consisting of SEQ ID NO: 5, and wherein the human growth hormone receptor antagonist has the amino acid sequence consisting of SEQ ID NO: 6. In certain implementations, the polyethylene glycol molecule contains a maleimide group for conjugation to a free sulfhydryl group. Certain implementations of the growth hormone receptor antagonist bind to the prolactin receptor. The disclosed composition may be used in a method for treating diseases or conditions responsive to growth hormone receptor antagonists by administering an effective amount of the disclosed composition.

A second disclosed composition provides a growth hormone receptor antagonist comprising human growth hormone receptor antagonist hGH-G120K having a DNA sequence of SEQ ID NO: 3 and an amino acid sequence of SEQ ID NO: 4, wherein a single amino acid of the human growth hormone receptor antagonist G120K has been mutated to cysteine, and wherein the single amino acid mutated to cysteine is T142; and a polydispersed 40 kDa branched polyethylene glycol molecule conjugated to the substituted cysteine in the human growth hormone receptor antagonist G120K mutant, and wherein the human growth hormone receptor antagonist is encoded by a DNA molecule having the sequence consisting of SEQ ID NO: 7, and wherein the human growth hormone receptor antagonist has the amino acid sequence consisting of SEQ ID NO: 8. In certain implementations, the polyethylene glycol molecule contains a maleimide group for conjugation to a free sulfhydryl group. Certain implementations of the growth hormone receptor antagonist bind to the prolactin receptor. Certain implementations of the growth hormone receptor antagonist exhibit about 20 times (20×) greater affinity for the human growth hormone receptor than pegvisomant. In certain implementations, the serum half-life of the growth hormone receptor antagonist is greater than the serum half-life of pegvisomant. In certain implementations, the serum half-life of the growth hormone receptor antagonist is 38 hours. The disclosed composition may be used in a method for treating diseases or conditions responsive to growth hormone receptor antagonists by administering an effective amount of the disclosed composition.

A third disclosed composition provides a growth hormone receptor antagonist comprising human growth hormone receptor antagonist hGH-G120K having a DNA sequence of SEQ ID NO: 3 and an amino acid sequence of SEQ ID NO: 4, wherein a single amino acid of the human growth hormone receptor antagonist G120K has been mutated to cysteine, and wherein the single amino acid mutated to cysteine is T142; and a polydispersed 40 kDa branched polyethylene glycol molecule conjugated to the substituted cysteine in the human growth hormone receptor antagonist G120K mutant, and wherein the human growth hormone receptor antagonist is encoded by a DNA molecule having the sequence consisting of SEQ ID NO: 9, and wherein the human growth hormone receptor antagonist has the amino acid sequence consisting of SEQ ID NO: 10. Certain implementations of the growth hormone receptor antagonist bind to the prolactin receptor. Certain implementations of the growth hormone receptor antagonist exhibit about 20 times (20×) greater affinity for the human growth hormone receptor than pegvisomant. In certain implementations, the serum half-life of the growth hormone receptor antagonist is greater than the serum half-life of pegvisomant. In certain implementations, the serum half-life of the growth hormone receptor antagonist is 38 hours. The disclosed composition may be used in a method for treating diseases or conditions responsive to growth hormone receptor antagonists by administering an effective amount of the disclosed composition. The disclosed composition may be used in a method for treating diseases or conditions responsive to growth hormone receptor antagonists by administering an effective amount of the disclosed composition.

It should be appreciated that all combinations of the foregoing concepts and additional concepts discussed in greater detail below (provided such concepts are not mutually inconsistent) are contemplated as being part of the technology disclosed herein and may be implemented to achieve the benefits as described herein. Additional features and aspects of the disclosed system, devices, and methods will become apparent to those of ordinary skill in the art upon reading and understanding the following detailed description of the example implementations. As will be appreciated by the skilled artisan, further implementations are possible without departing from the scope and spirit of what is disclosed herein. Accordingly, the descriptions provided herein are to be regarded as illustrative and not restrictive in nature.

### BRIEF DESCRIPTION OF THE DRAWINGS

The accompanying drawings, which are incorporated into and form a part of the specification, schematically illustrate one or more example implementations of the disclosed technology and together with the general description given above and detailed description given below, serve to explain the principles of the disclosed subject matter, and wherein:

FIG. 1 is a photograph of an SDS PAGE analysis of purified Compound D₂ (M-hGH-G120K-T142C-GL2-400MA) showing a single broad band slightly below the 100 kDa molecular weight standard, wherein lane 1 includes Compound D₂ and wherein lane 2 includes molecular weight standards;

FIG. 2 depicts MALDI-TOF mass spectra of Compound D₂ showing a single major [M+H]⁺ peak;

FIG. 3 is a plot of the assay response versus GHRA concentration for the inhibition of Biotin-hGH binding to the GHR by hGH, Compound D₂, and pegvisomant; and

FIG. 4 is a plot of the assay response versus GHRA concentration for the inhibition of Biotin-hGH binding to the PRL receptor by hGH, Compound D₂, and pegvisomant.

### DETAILED DESCRIPTION

Example implementations are now described with reference to the Figures. Reference numerals are used throughout the detailed description to refer to the various elements and structures. Although the following detailed description contains many specifics for the purposes of illustration, a person of ordinary skill in the art will appreciate that many variations and alterations to the following details are within the scope of the disclosed technology. Accordingly, the following implementations are set forth without any loss of generality to, and without imposing limitations upon, the claimed subject matter.

The examples discussed herein are examples only and are provided to assist in the explanation of the apparatuses, devices, systems, and methods described herein. None of the features or components shown in the drawings or discussed below should be taken as required for any specific implementation of any of these the apparatuses, devices, systems or methods unless specifically designated as such. For ease of reading and clarity, certain components, modules, or methods may be described solely in connection with a specific Figure. Any failure to specifically describe a combination or sub-combination of components should not be understood as an indication that any combination or sub-combination is not possible. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

The disclosed technology provides various novel human growth hormone (hGH) antagonists for use primarily as therapeutics. U.S. Pat. No. 10,874,717, which is incorporated by reference herein in its entirety for all purposes, discloses the preparation of hGH antagonist molecules that are pegylated in predetermined positions. The hGH antagonists of this invention are typically made by mutating one or more selected amino acids of hGH G120K, a known hGH antagonist, to cysteines and then conjugating the cysteines to chemically activated polyethylene glycol molecules. The positions of the various substituted cysteines were selected for minimal loss in hGH receptor binding activity after conjugation with polyethylene glycol. The size and the number of PEGs added were selected to prevent filtration of these molecules in the kidneys, thereby prolonging in vivo half-lives.

Two important variables in the preparation of the disclosed hGH antagonists include: (i) the amino acid position used for PEG attachment; and (ii) the size and type of the conjugated PEG. Initial research with similar compositions was done using random attachment of relatively small PEGs (e.g., about 5 kDa) to multiple lysines on the surfaces of proteins. This procedure successfully increased the in vivo half-lives of the proteins but resulted in large decreases in the affinity of the proteins for their receptors. More recent experimental approaches have added PEG molecules to specific amino acid sites on proteins. Two common methods used for site specific Pegylation are: (i) addition of PEG to the N-terminal amine of proteins by way of low pH reductive amination; and (ii) addition of PEG to the thiol groups of cysteines that are either native to the protein or engineered into specific positions. Other methods include PEG addition to unnatural amino acids; PEG addition to proteins C-termini by way of intein fusion proteins; and PEG addition to accessible glutamines by way of transaminase catalysis.

As discussed in detail in U.S. Pat. No. 10,874,717, two different types or classes of polyethylene glycol (PEG) molecules may be utilized with the disclosed technology. The first class of PEGs was prepared by polymerization and is useful for modifying proteins to increase their in vivo half-lives. This type of PEG is by nature polydispersed, meaning that there is a distribution of molecular weight products around the average molecular weight. The PEGs include a 20 kDa linear PEG (Layson Bio, MPEG-MAL-20,000), a 40 kDa branched PEG (NOF, Sunbright GL2-400MA), and a linear 40 kDa PEG (NOF, Sunbright ME-400MA). These PEGS each contain a maleimide group for conjugation to the free sulfhydryl groups of the mutant proteins. The second class of polyethylene glycols are DPEGS® (discrete polyethylene glycols) (Quanta BioDesign). These DPEGS® (discrete polyethylene glycols) are pure single PEG molecules that are prepared using stepwise, organic chemistry so that each DPEGS® (discrete polyethylene glycols) species is a pure single compound with a specific structure and molecular weight. The DPEGS® (discrete polyethylene glycols) used in this invention, which typically contain a maleimide group for coupling to free thiols, may include the following: a tri-branched molecule with a molecular weight of 4473 Daltons and a carboxylate anion at the terminus of each branch (Quanta BioDesign #10451, MAL-DPEG®A); a neutral tri-branched molecule with a molecular weight of 4299 Daltons (Quanta BioDesign #4229, MAL-DPEG®B); a neutral 9-branched molecule with a molecular weight of 8324 (Quanta Biodesign #10484; MAL-DPEG®E); and a neutral 9-branched molecule with a molecular weight of 15,592 (Quanta Biodesign #11487; MAL-DPEG®F).

The disclosed compositions provide novel human growth hormone receptor antagonists that are useful in various therapeutic applications. TABLE 1, below, includes names and abbreviations for specific human growth hormone (hGH) antagonists disclosed herein. For reference purposes, SEQ ID NO: 1 provides the DNA sequence for human growth hormone WThGH and SEQ ID NO: 2 and provides the amino acid sequence for human growth hormone WThGH (mature form), referred to herein as hGH. Human growth hormone receptor antagonist hGH-G120K, referred to herein as G120K, is the parent receptor antagonist for the compositions of the disclosed technology, and for reference purposes, SEQ ID NO: 3 provides the DNA sequence for human growth hormone receptor antagonist G120K and SEQ ID NO: 4 provides the amino acid sequence for human growth hormone receptor antagonist G120K (mature form). A first disclosed pegylated growth hormone receptor antagonist having an increased serum half-life is GGSSG-hGH-G120K-T142C-GL2-400MA, referred to herein as Compound D₁, and for reference purposes, SEQ ID NO: 5 provides the DNA sequence for Compound D₁ and SEQ ID NO: 6 provides the amino acid sequence for Compound D₁. A second disclosed pegylated growth hormone receptor antagonist having an increased serum half-life is M-hGH-G120K-T142C-GL2-400MA, referred to herein as Compound D₂, and for reference purposes, SEQ ID NO: 7 provides the DNA sequence for Compound D₂ and SEQ ID NO: 8 provides the amino acid sequence for Compound D₂. A third disclosed pegylated growth hormone receptor antagonist having an increased serum half-life is hGH-G120K-T142C-GL2-400MA, referred to herein as Compound D₃, and for reference purposes, SEQ ID NO: 9 provides the DNA sequence for Compound D₃ and SEQ ID NO: 10 provides the amino acid sequence for Compound D₃. The single letter amino acid abbreviations used herein follow the IUPAC format.

TABLE 1
Designations for hGH and Mutant hGH Antagonists

| Name | Abbreviation | SED ID NOS |
|---|---|---|
| hGH | hGH | 1-2 |
| G120K | hGH-G120K | 3-4 |
| Compound D₁ | GGSSG-hGH-G120K-T142C- | 5-6 |
| | GL2-400MA | |
| Compound D₂ | M-hGH-G120K-T142C-GL2-400MA | 7-8 |
| Compound D₃ | hGH-G120K-T142C-GL2-400MA | 9-10 |
| Compound G₂ | M-hGH-G120K-T142C-dPEGA- | 11-12 |
| | H151C-dPEGA | |

The preparation of Compound D₁ (GGSSG-hGH-G120K-T142C-GL2-400MA) is disclosed in U.S. Pat. No. 10,874,717. The preparation of Compound D₂ (M-hGH-G120K-T142C-GL2-400MA) is disclosed below. The method disclosed is also relevant with regarding to preparing Compound D₃ (hGH-G120K-T142C-GL2-400MA; however, other methods for preparing Compound D₃ are possible and are utilized in alternate implementations.

With reference to TABLE 1, the three disclosed Compound D variants differ in the amino acids that are attached to the phenylalanine (F) that is the N-terminus of native hGH. Compound D₁ includes the sequence GGSSGF- on its N-terminus (see SEQ ID NO: 13), Compound D₂ includes an ME-on its N-terminus, and Compound D₃ begins with the native phenylalanine (F-). The affinities of D₁ and D₂ for the hGH receptor are essentially identical despite their differing by five N-terminal amino acids and are similar to the affinity of native hGH for the receptor. Variant D₃, which differs from D₂ by only a single amino acid will presumably exhibit a similar affinity for hGH receptor.

Compound D₂ contains a non-native an N-terminal methionine. Because the methionine is adjacent to a phenylalanine, the N-terminal amino acid of native growth hormone, it is not readily cleaved by the N-terminal methionine aminopeptidase of E. coli. However, the preparation of the antagonist without the methionine (D₃), having utility as a therapeutic, can be readily performed one of ordinary skill in the art. Compound D₃ can be produced without the N-terminal methionine by expression into the E. coli periplasm [14], expression into the cell medium [15], or expression by mammalian cells [16]. Compound G₁ (GGSSG-hGH-G120K-H151C-T142C-dPEGA₂), which is disclosed in U.S. Pat. No. 10,874,717, can be made without the GGSSG terminus by these published methods.

### I. Preparation of Compound-D₂ (M-hGH-G120K-T142C-GL2-400MA)

### Gene Construction for Compound D₂

The gene for Compound D₂ (M-hGH-G120K-T142C-GL2-400MA) was synthesized and cloned into a pET-21a vector by Genscript (Piscataway, NJ). The complete DNA sequence of this molecule is SEQ ID NO. 7 and the complete amino acid sequence of this molecule is SEQ ID NO 8. The construct was transformed into Lemos (DE3) (New England Biolabs, Ipswich, MA) for protein expression. The two site-specific substitutions are G120K and T142C.

### Protein Expression

A primary culture was prepared by inoculating 2 mL of LB broth containing 100 μg/mL of carbenicillin with a frozen glycerol stock of E. coli containing the M-hGH-G120K-T142C plasmid. The inoculated broth was incubated at 37° C. for 4 hours with shaking at 200 RPM. This culture was then used to inoculate 30 mL of LB broth, containing 100 μg/mL carbenicillin, and incubated overnight at 37° C. with shaking at 150 RPM. Four 2.5 L flasks, each containing 750 mL of TB broth and carbenicillin (100 μg/mL), were inoculated with 7.5 mL of the overnight culture and incubated for 2 hours 45 minutes, at which time the A (600) nm of the cultures reached 0.6-1.0 OD. The cultures were then cooled to 16° C., made 1 mM isopropyl β-D-1-thiogalactopyranoside, and incubated overnight with shaking at 200 RPM to induce expression. The cultures were then harvested by centrifugation at 6080 rcf for 15 minutes at 4° C., resulting in cell pellets that were stored at −20° C. until purification.

### Cell Disruption

Cell pellets from 1.5 L batches of culture were suspended by vortexing in 150 mL of cold PBS containing 5 mM cysteine and 0.1 mL of a protease inhibitor cocktail (Sigma P8849). The suspended cells were cooled in an ice water bath and sonicated (Fisher 100 sonic dismembrator, 5 mm probe tip) for 30 seconds followed by a 5-minute incubation to cool the solution. The sequence of sonication followed by cooling was repeated five more times for a total of six sonication cycles. The samples were then centrifuged for 30 minutes at 30,600 rcf at 4° C.

### Ammonium Sulfate Precipitation

The supernatant from the centrifuged sonicate was stirred at 4° C. and made 1.3 M ammonium sulfate by the addition of cold 3 M ammonium sulfate in PBS, pH 7. After stirring for 15 minutes at 4° C., the precipitate was pelleted by centrifugation at 4° C. for 30 minutes at 30,600 rcf.

### Glutathione Reduction

The ammonium sulfate precipitate was re-dissolved in 100 mL of cold PBS by gentle stirring at 4° C. for 30 minutes. The solution was then made 1.5 mM reduced glutathione, incubated for 2 hours at RT, and concentrated to 50 mL using a 10 kDa concentrator. The concentrated sample was applied at RT to a 1 L Sephadex G-25 column that was equilibrated with aerated PBS to remove the glutathione and allow oxidation of the native cysteines of the M-hGH-G120K mutant to form the native disulfide bonds.

### Pegylation

The pegylation reaction requires that the two native disulfide bonds of hGH be correctly formed and that the cysteine substituted at position T142 is reduced. After removal of the glutathione, the oxidation state of the cysteines was determined by reacting small aliquots of the product with 0.2 mM of a maleimide activated, 40 kDa 2-branched PEG (SUNBRIGHT GL2-400MA, NOF America). After incubation for 1 hour at room temperature, the samples were analyzed by SDS PAGE. In cases when the native cysteines were not fully oxidized, the gels showed bands with lower mobilities (higher MW) than the mobility of Compound D₂, caused by pegylating both the native cysteines and the cysteine substituted for threonine at position 142. If the gel showed that only a single PEG had been added, then a large scale pegylation was performed with the 40 kDa maleimide activated PEG, as described below. If higher molecular weight bands appeared, then the formation of the native disulfides was catalyzed by making the reaction 2.5 μM copper sulfate and incubating at room temperature for 10 min. A small aliquot was reacted with the 40 kDa 2-branched PEG as described above and analyzed by SDS-PAGE to ensure that the native disulfide bonds had formed.

When the native disulfide bonds had formed, the sample, which was in pH 7.4 PBS, was adjusted to 1 mg/mL protein (45 μM) and a maleimide activated 40 kDa 2-branched PEG (SUNBRIGHT GL2-400MA, NOF America) was added to a final concentration of 50 μM. The reaction was allowed to proceed at RT for 1 hour and then stored at 4° C. prior to purification.

### Size Exclusion Chromatography

Compound D₂ was concentrated on a 10 kDa centrifugal concentrator to ˜10 mg/mL and then aliquots of 10-15 mL were applied to a HiLoad Superdex 200 pg size exclusion column at room temperature (Cytiva; 26 mm×600 mm) that was equilibrated in filter sterilized TBS, pH 8 containing 10% glycerol. The SEC buffer was made just prior to use with endotoxin free water. Fractions that contained Compound D₂ were identified by SDS-PAGE and combined.

### Antion Exchange Chromatography

All buffers for anion exchange chromatography were made using endotoxin free water. The combined SEC fractions were concentrated to ˜10 mg/mL on a 10 kDa centrifugal concentrator (Amicon Ultra 15) and then diluted to 1 mg/mL with 5 mM Tris buffer, pH 8. The sequence of concentration and dilution was repeated two additional times and the conductivity was measured. If the conductivity was <0.5 mSiemens, then the sample was purified by anion exchange. If not, then the sequence of concentration and dilution was repeated until a conductivity level of less than 0.5 mSiemens achieved. The product was then purified and endotoxin removed using anion exchange chromatography at 4° C. on a column consisting of five 5-mL HiTrap Q Sepharose FF anion exchange columns (Cytiva) connected together. The column was equilibrated in 5 mM Tris, pH 8 and the product, which was in a low salt pH 8 Tris buffer, was then applied to the column that was then washed with the following sequence of buffers at 4° C.: 5 CV 5 mM Tris, pH 8; 30 CV 0.1% Triton X-114 in 5 mM Tris, pH 8; 10 CV 1% CHAPS in 5 mM Tris, pH 8; and 5 CV 5 mM Tris, pH 8. The column was then eluted stepwise with a 5 mM Tris, pH 8 buffer containing different concentrations of sodium chloride; 2 CV of 12.5 mM sodium chloride, 5 CV of 25 mM sodium chloride, 10 CV of 50 mM sodium chloride and 10 CV of 100 mM sodium chloride. Fractions (6 mL) were collected and 0.3 mL 20×PBS was added to each fraction. The majority of the product eluted in the buffer containing 50 mM NaCl. Fractions that contained pure product were identified by SDS-PAGE. These fractions were collected, and the buffer was exchanged for PBS using the concentration/dilution procedure described above. The final product was found to contain <5 EU endotoxin/mg protein using the Pierce Chromatogenic Endotoxin Quantitation Kit (S39552).

### Compound D₂ Characterization

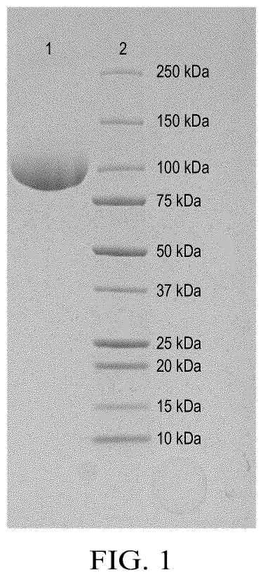

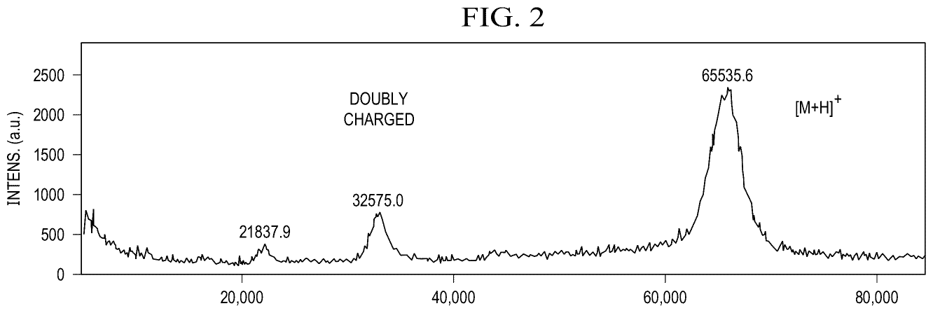

FIG. 1 is a photograph of an SDS PAGE analysis of purified Compound D₂ (M-hGH-G120K-T142C-GL2-400MA) showing a single broad band slightly below the 100 kDa molecular weight standard, wherein lane 1 includes Compound D₂ and wherein lane 2 includes molecular weight standards. FIG. 2 depicts MALDI-TOF mass spectra of Compound D₂ showing a single major [M+H]⁺ peak. The experimental [M+H]⁺ MALDI-TOF MW is 65.5 kDa while the calculated MW is 63.3 kDa. The difference between the experimental and calculated molecular weights for Compound D₂ is due to the polydispersed nature of the 40 kDa PEG, resulting in a very broad MALDI-TOF peak.

### II. Receptor Binding Properties

The relative affinities of the growth hormone receptor antagonists (GHRAs) were determined using the methods disclosed in U.S. Pat. No. 10,874,717. Microtiter plates were coated with the extracellular domain of the growth hormone receptor and the abilities of the different GHRAs to inhibit the binding of biotin-hGH to the receptor were determined as a function of GHRA concentration. From these experiments, the concentration of unlabeled hGH, Compound D₂, and pegvisomant that inhibited 50% of the binding of biotin-hGH to the receptor (IC50) were determined. The IC50 is inversely proportional to the affinity of the GHRA for the receptor.

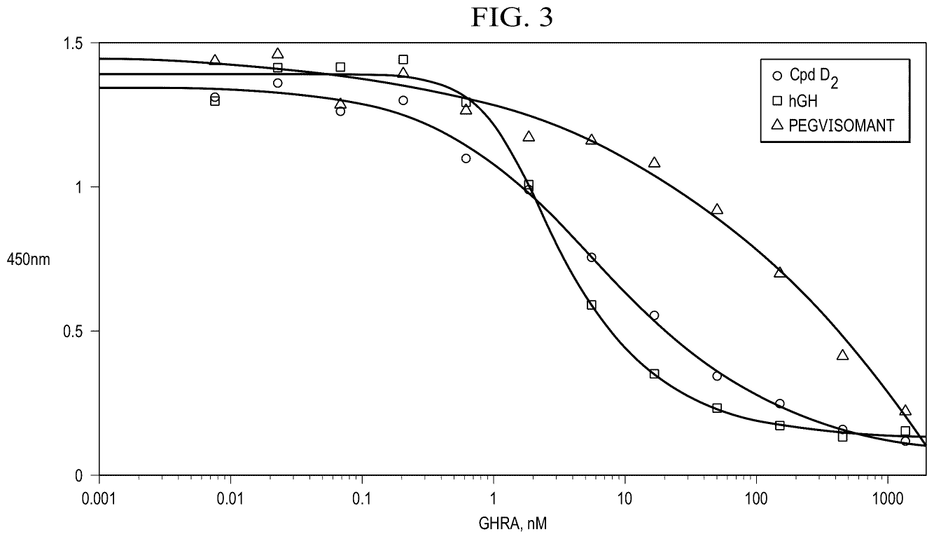

FIG. 3 is a plot of an inhibition assay response versus GHRA concentration for the inhibition of Biotin-hGH binding to the GHR by hGH, Compound D₂, and pegvisomant. From this plot, the IC50 values for hGH (3.6 nM), Compound D₂ (6.6 nM), and pegvisomant (137 nM) were determined. The affinities of the GHRAs for the growth hormone receptor relative to the affinity of hGH can be calculated from the IC50 of hGH divided by the IC50 of the GHRA. As shown in TABLE 2, below, the relative affinities of Compound D₂ and pegvisomant for the receptor are 0.55 and 0.026 respectively.

TABLE 2
IC50 Values for the Inhibition of Biotin-hGH
Binding to the GHR Coated on an ELISA Plate

| | IC50 | IC50 hGH |
|---|---|---|
| Compound | nM | IC50 GHRA |
| hGH | 3.6 | 1.0 (100%) |
| Compound D₂ | 6.6 | 0.55 (55%) |
| pegvisomant | 137 | 0.026 (2.6%) |

The data in TABLE 2 show that growth hormone binds to the growth hormone receptor ˜40× more tightly than pegvisomant. This result is similar to the result found in the literature where hGH bound to hGHR on cell surfaces and to the hGHR on isolated membranes ˜40× and ˜30× more tightly than pegvisomant (B2036-PEG) respectively. These results also show that Compound D₂ binds to the GHR ˜20× more tightly than does pegvisomant, indicating that Compound D₂ is a likely to be a highly effective therapeutic for any indication that requires inhibition of the GHR.

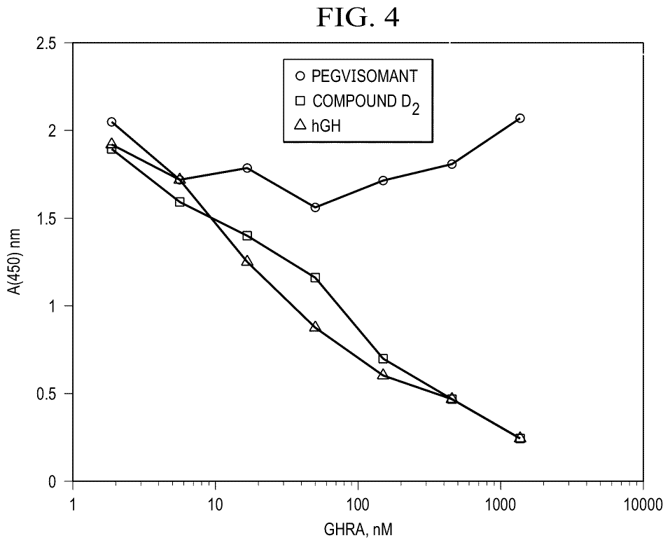

Pegvisomant, which comprises hGH-G120K and eight (8) additional mutations, has four to six 5 kDa linear PEG molecules attached to random lysine residues. The eight additional mutations increase drug affinity for the soluble growth hormone receptor and remove its affinity for the prolactin receptor (Ross, 2001). Pegvisomant is an effective treatment for acromegaly even though its randomly conjugated PEG molecules reduce its affinity for the hGHR by about 30 to 40 fold relative to hGH [10]. The hGH antagonists disclosed herein only reduce the receptor affinity about 2-fold, making these compounds potentially more effective as treatment for acromegaly. In contrast to pegvisomant, the disclosed antagonists bind to the prolactin receptor with affinities similar to that of hGH (See FIG. 4, which is a plot of the assay response versus GHRA concentration for the inhibition of Biotin-hGH binding to the PRL receptor by hGH, Compound D₂, and pegvisomant).

### III. Serum Half Life

Three-month-old C57BL/6J male mice (n=3 for each compound, Jackson Lab) were treated with a single intraperitoneal injection of 200 mg/kg of Compound D₂, pegvisomant, or Compound G2 and blood was periodically collected to measure changes in plasma levels of the three compounds at 0-h (pre-injection), and at 6-h, 24-h, 48-h, 72-h, and 96-h post-injection. The plasma levels for the dosed compounds were analyzed using sandwich ELISA assays.

The T_{1/2} values, calculated using WinNonlin software, are shown in TABLE 3. The half-life for Compound D₂ is 38 hours compared to a half-life of 30 hours for pegvisomant and 14 hours for Compound G2. With both a longer half-life and significantly better receptor binding, Compound D₂ is presumably a more effective therapeutic for disease indications that can be treated by inhibiting the hGH receptor.

TABLE 3
Half Life for Pegvisomant Compound D₂ and Compound
G₂ after Intraperitoneal injection into Mice

| Compound | | T_{1/2} |
|---|---|---|
| Compound D₂ | Mean | 38 |
| | SD | 4 |
| Pegvisomant | Mean | 32 |
| | SD | 10 |
| Compound G₂ | Mean | 14 |
| | SD | 6 |

Compound D₃ is presumably a more effective growth hormone receptor antagonist for treatment of cancer than pegvisomant because Compound D₂, which is a very close analog of compound D₃, exhibits a 20 fold higher affinity for hGH receptor, as well as a longer half-life, and the ability to bind to the prolactin receptor. Compound D₃ is presumably a more effective growth hormone receptor antagonist for treatment of cancer than Compound G3 (see U.S. Pat. No. 10,874,717) based on the longer observed half-life of D₂, which is a very close analog of compound D₃ (T½ D₂=38 hrs; T½ G₂=14 hrs).

Consistent with U.S. Pat. Nos. 10,874,717 and 11,452,763, both of which are expressly incorporated-by-reference herein in their entirety for all purposes, the disclosed compositions are useful as therapeutics for various diseases or conditions responsive to human growth hormone receptor antagonists. These diseases or conditions may include acromegaly and cancers that express high levels of the growth hormone receptor; high levels of the prolactin receptor; or high levels of both the growth hormone receptor and the prolactin receptor. The cancers include breast cancer, central nervous system cancer, melanoma, non-small cell lung cancer, ovarian cancer, prostate cancer, and renal cancer.

All literature and similar material cited in this application, including, but not limited to, patents, patent applications, articles, books, treatises, and web pages, regardless of the format of such literature and similar materials, are expressly incorporated by reference in their entirety. Should one or more of the incorporated references and similar materials differ from or contradict this application, including but not limited to defined terms, term usage, described techniques, or the like, this application controls.

As previously stated and as used herein, the singular forms “a,” “an,” and “the,” refer to both the singular as well as plural, unless the context clearly indicates otherwise. The term “comprising” as used herein is synonymous with “including,” “containing,” or “characterized by,” and is inclusive or open-ended and does not exclude additional, unrecited elements or method steps. Although many methods and materials similar or equivalent to those described herein can be used, particular suitable methods and materials are described herein. Unless context indicates otherwise, the recitations of numerical ranges by endpoints include all numbers subsumed within that range. Furthermore, references to “one implementation” are not intended to be interpreted as excluding the existence of additional implementations that also incorporate the recited features. Moreover, unless explicitly stated to the contrary, implementations “comprising” or “having” an element or a plurality of elements having a particular property may include additional elements whether or not they have that property.

The terms “substantially” and “about”, if or when used throughout this specification describe and account for small fluctuations, such as due to variations in processing. For example, these terms can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%, and/or 0%.

Underlined and/or italicized headings and subheadings are used for convenience only, do not limit the disclosed subject matter, and are not referred to in connection with the interpretation of the description of the disclosed subject matter. All structural and functional equivalents to the elements of the various implementations described throughout this disclosure that are known or later come to be known to those of ordinary skill in the art are expressly incorporated herein by reference and intended to be encompassed by the disclosed subject matter. Moreover, nothing disclosed herein is intended to be dedicated to the public regardless of whether such disclosure is explicitly recited in the above description.

There may be many alternate ways to implement the disclosed technology. Various functions and elements described herein may be partitioned differently from those shown without departing from the scope of the disclosed technology. Generic principles defined herein may be applied to other implementations. Different numbers of a given module or unit may be employed, a different type or types of a given module or unit may be employed, a given module or unit may be added, or a given module or unit may be omitted.

Regarding this disclosure, the term “a plurality of” refers to two or more than two. Unless otherwise clearly defined, orientation or positional relations indicated by terms such as “upper” and “lower” are based on the orientation or positional relations as shown in the Figures, only for facilitating description of the disclosed technology and simplifying the description, rather than indicating or implying that the referred devices or elements must be in a particular orientation or constructed or operated in the particular orientation, and therefore they should not be construed as limiting the disclosed technology. The terms “connected”, “mounted”, “fixed”, etc. should be understood in a broad sense. For example, “connected” may be a fixed connection, a detachable connection, or an integral connection, a direct connection, or an indirect connection through an intermediate medium. For one of ordinary skill in the art, the specific meaning of the above terms in the disclosed technology may be understood according to specific circumstances.

It should be appreciated that all combinations of the foregoing concepts and additional concepts discussed in greater detail herein (provided such concepts are not mutually inconsistent) are contemplated as being part of the disclosed technology. In particular, all combinations of claimed subject matter appearing at the end of this disclosure are contemplated as being part of the technology disclosed herein. While the disclosed technology has been illustrated by the description of example implementations, and while the example implementations have been described in certain detail, there is no intention to restrict or in any way limit the scope of the appended claims to such detail. Additional advantages and modifications will readily appear to those skilled in the art. Therefore, the disclosed technology in its broader aspects is not limited to any of the specific details, representative devices and methods, and/or illustrative examples shown and described. Accordingly, departures may be made from such details without departing from the spirit or scope of the general inventive concept.

The following references form part of the specification of the present application and each reference is incorporated by reference herein, in its entirety, for all purposes.
1. Pasut, G. and Veronese, M. (2012) State of the Art in Pegylation: The Great Versatility Achieved After Forty Years of Research. J. Controlled Release 161, 461-472.
2. Parveen, S. and Sahoo, S. K. Nanomedicine: Clinical Applications of Polyethylene Glycol Conjugated to Proteins and Drugs Clin. Pharmacokinet. 45, 965-988.
3. Alconcel, S. N. S., Baas, A. S. and Maynard, H. D. (2011) FDA-Approved Poly (ethylene glycol)-Protein Conjugate Drugs. Polymer Chemistry 2, 1442-1448.
4. Kling, J. (2013) Pegylation of Biologics: A Multipurpose Solution. Bioprocess International 11, 35-43.
5. Sustarsic, E. G., Junnila, R. K., and Kopchick, J. J. (2013) “Human Metastatic Melanoma Cell Lines Express High Levels of Growth Hormone Receptor and Respond to GH Treatment” Biochem Biophys Res Commun. 441:144-150.
6. Basu, R., Wu, S., and Kopchick, J. J. (2017-1) “Targeting Growth Hormone Receptor in Human Melanoma Cells Attenuates Tumor Progression and Epithelial Mesenchymal Transition Via Suppression of Multiple Oncogenic Pathways” Oncotarget 8, 21579-21598.
7. Basu, R., Baumgaertel, N., Wu, S., and Kopchick, J. J. (2017-2) “Growth Hormone Receptor Knockdown Sensitizes Human Melanoma Cells to Chemotherapy by Attenuating Expression of ABC Drug Efflux Pumps” Horm. Canc. 8, 143-156.
8. Xu, J., Sun, D., Jiang, J., Deng., L., Zhang, Y., Yu, H., Bahl, D., Langenheim, J. F., Chen, W. Y., Fuchs, S. Y., and Frank, S. J. (2013) “The Role of Prolactin Receptor in GH Signaling in Breast Cancer Cells” Mol. Endocrinol, 27, 266-279.
9. Goffin, V., Bernichtein, S., Carriere, O., Bennet, W. F., Kopchick, J. J., and Kelly, P. A. (1999) Endocrinology 140, 3853-3856.
10. Ross, R. J. M. et al. “Binding and Functional Studies with the Growth Hormone Receptor Antagonist B2036-PEG (Pegvisomant) Reveal Effects of Pegylation and Evidence that it Binds to a Receptor Dimer” The Journal of Clinical Endocrinology & Metabolism 86.4 (2001) 1716-1723.
11. Zheng-Sheng Wu, Kun Yang, Yu Wan, Peng-Xu Qian, Jo K Perry, Jean Chiesa, Hichem C Mertani, Tao Zhu, Peter E Lobie (2011) Tumor expression of human growth hormone and human prolactin predict a worse survival outcome in patients with mammary or endometrial carcinoma. J. Clin. Endocrinol. Metab. 96, E1619-1629.
12. Schuler, L. A., and O'Leary, K. A. (2022) Prolactin: the third hormone in breast cancer. Front Endocrinol. (Lausanne) 13, 910978.
13. Reetobrata Basu, Rich Brody, Uday Sandbhor, Prateek Kulkarni, Emily Davis, Deborah Swegan, Lydia J. Caggiano, Edward Brenya, Sebastian Neggers, and John J. Kopchick (2023) Structure and function of a dual antagonist of the human growth hormone and prolactin receptors with site-specific PEG conjugates. J. Biol. Chem. 299 (8) 105030.
14 Ezequiel et al., High-yield purification of biosynthetic human growth hormone secreted in Escherichia coli periplasmic space” J. Chromat. A. (1999) 852, 441-450.
15 Gigi et al., “Optimization of expression, purification and secretion of functional recombinant human growth hormone in Escherichia coli using modified staphylococcal protein a signal peptide” (2021) BMC Biotechnology 21:51.
16. Nottorf et al., “Production of Human Growth Hormone in a Mammalian Cell High Density Perfusion Process” (2007) In: Smith, R. (eds) Cell Technology for Cell Products., vol 3. Springer, Dordrecht. https://doi.org/10.1007/978-1-4020-5476-1_145.

What is claimed:

1. A growth hormone receptor antagonist, comprising:
(a) human growth hormone receptor antagonist G120K having a DNA sequence of SEQ ID NO: 3 and an amino acid sequence of SEQ ID NO: 4, wherein a single amino acid of the human growth hormone receptor antagonist G120K has been mutated to cysteine, and wherein the single amino acid mutated to cysteine is T142; and
(b) a polydispersed 40 kDa branched polyethylene glycol molecule conjugated to the substituted cysteine in the human growth hormone receptor antagonist G120K mutant, and
(c) wherein the human growth hormone receptor antagonist is encoded by a DNA molecule having the sequence consisting of SEQ ID NO: 5, and wherein the human growth hormone receptor antagonist has the amino acid sequence consisting of SEQ ID NO: 6.

2. The composition of claim 1, wherein the polyethylene glycol molecule contains a maleimide group for conjugation to a free sulfhydryl group.

3. The composition of claim 1, wherein the growth hormone receptor antagonist is configured to bind to the prolactin receptor.

4. A method for treating diseases or conditions responsive to growth hormone receptor antagonists, comprising administering an effective amount of the composition of claim 1.

5. A growth hormone receptor antagonist, comprising:
(a) human growth hormone receptor antagonist G120K having a DNA sequence of SEQ ID NO: 3 and an amino acid sequence of SEQ ID NO: 4, wherein a single amino acid of the human growth hormone receptor antagonist G120K has been mutated to cysteine, and wherein the single amino acid mutated to cysteine is T142; and
(b) a polydispersed 40 kDa branched polyethylene glycol molecule conjugated to the substituted cysteine in the human growth hormone receptor antagonist G120K mutant, and
(c) wherein the human growth hormone receptor antagonist is encoded by a DNA molecule having the sequence consisting of SEQ ID NO: 7, and wherein the human growth hormone receptor antagonist has the amino acid sequence consisting of SEQ ID NO: 8.

6. The composition of claim 5, wherein the polyethylene glycol molecule contains a maleimide group for conjugation to a free sulfhydryl group.

7. The composition of claim 5, wherein the growth hormone receptor antagonist is configured to bind to the prolactin receptor.

8. The composition of claim 5, wherein the growth hormone receptor antagonist exhibits about 20 times greater affinity for a human growth hormone receptor than pegvisomant.

9. The composition of claim 5, wherein the serum half-life of the growth hormone receptor antagonist is greater than the serum half-life of pegvisomant.

10. The composition of claim 5, wherein the serum half-life of the growth hormone receptor antagonist is 38 hours.

11. A method for treating diseases or conditions responsive to growth hormone receptor antagonists, comprising administering an effective amount of the composition of claim 5.

12. A growth hormone receptor antagonist, comprising:
(a) human growth hormone receptor antagonist G120K having a DNA sequence of SEQ ID NO: 3 and an amino acid sequence of SEQ ID NO: 4, wherein a single amino acid of the human growth hormone receptor antagonist G120K has been mutated to cysteine, and wherein the single amino acid mutated to cysteine is T142; and
(b) a polydispersed 40 kDa branched polyethylene glycol molecule conjugated to the substituted cysteine in the human growth hormone receptor antagonist G120K mutant, and
(c) wherein the human growth hormone receptor antagonist is encoded by a DNA molecule having the sequence consisting of SEQ ID NO: 9, and wherein the human growth hormone receptor antagonist has the amino acid sequence consisting of SEQ ID NO: 10.

13. The composition of claim 12, wherein the polyethylene glycol molecule contains a maleimide group for conjugation to a free sulfhydryl group.

14. The composition of claim 12, wherein the growth hormone receptor antagonist is configured to bind to the prolactin receptor.

15. The composition of claim 12, wherein the growth hormone receptor antagonist exhibits about 20 times greater affinity for a human growth hormone receptor than pegvisomant.

16. The composition of claim 12, wherein the serum half-life of the growth hormone receptor antagonist is greater than the serum half-life of pegvisomant.

17. The composition of claim 12, wherein the serum half-life of the growth hormone receptor antagonist is 38 hours.

18. A method for treating diseases or conditions responsive to growth hormone receptor antagonists, comprising administering an effective amount of the composition of claim 12.


SEQUENCE LISTING (13 sequences)

SEQ ID NO: 1 - DNA, 576 bp, synthetic construct
  source 1..576: mol_type = other DNA
ttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 2 - protein, 191 aa, synthetic construct [sequence omitted]

SEQ ID NO: 3 - DNA, 576 bp, synthetic construct
  source 1..576: mol_type = other DNA
ttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 4 - protein, 191 aa, synthetic construct
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQTYSKFDTNSHNDDALLKNYGLLYCFRKDMDKVETFLRIVQCRSVEGSCGF

SEQ ID NO: 5 - DNA, 591 bp, synthetic construct
  source 1..591: mol_type = other DNA
ggtggaagctcgggattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagtgctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 6 - protein, 196 aa, synthetic construct
GGSSGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQCYSKFDTNSHNDDALLKNYGLLYCFRKDMDKVETFLRIVQCRSVEGSCGF

SEQ ID NO: 7 - DNA, 579 bp, synthetic construct
  source 1..579: mol_type = other DNA
atgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagtgctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 8 - protein, 192 aa, synthetic construct
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQCYSKFDTNSHNDDALLKNYGLLYCFRKDMDKVETFLRIVQCRSVEGSCGF

SEQ ID NO: 9 - DNA, 576 bp, synthetic construct
  source 1..576: mol_type = other DNA
ttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagtgctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 10 - protein, 191 aa, synthetic construct
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQCYSKFDTNSHNDDALLKNYGLLYCFRKDMDKVETFLRIVQCRSVEGSCGF

SEQ ID NO: 11 - DNA, 579 bp, synthetic construct
  source 1..579: mol_type = other DNA
atgttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaaagatccaaacgctgatggggaggctggaagatggcagcccccggactgggcagatcttcaagcagtgctacagcaagttcgacacaaactcatgcaacgatgacgcactactcaagaactacgggctgctctactgcttcaggaaggacatggacaaggtcgagacattcctgcgcatcgtgcagtgccgctctgtggagggcagctgtggcttctag

SEQ ID NO: 12 - protein, 192 aa, synthetic construct
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQCYSKFDTNSCNDDALLKNYGLLYCFRKDMDKVETFLRIVQCRSVEGSCGF

SEQ ID NO: 13 - protein, 5 aa, synthetic construct
GGSSG